(19) United States
(12) Patent Application Publication
(10) Pub. No.: US 2025/0125029 A1
(43) Pub. Date: Apr. 17, 2025

(54) INTERNET OF THINGS (IOT) BASED EMERGENCY MEDICAL EVENT MANAGEMENT SYSTEM AND METHOD FOR IDENTIFYING EMERGENCY MEDICAL EVENTS AND PROVIDING RESCUE MEDICATION DURING EMERGENCY MEDICAL EVENTS

(71) Applicant: Auriva Health, Inc., New Hartford, NY (US)

(72) Inventors: Jolene Bressi, New Hartford, NY (US); Melinda Chu, Blue Ash, OH (US)

(21) Appl. No.: 18/918,175

(22) Filed: Oct. 17, 2024

Related U.S. Application Data
(60) Provisional application No. 63/590,791, filed on Oct. 17, 2023.

(51) Int. Cl. G16H 20/10; A61B 5/00; G16H 10/60; G16H 40/67; G16Y 10/60; G16Y 20/40

(52) U.S. Cl. CPC G16H 20/10; A61B 5/0022; A61B 5/7221; A61B 5/7282; G16H 10/60; G16H 40/67; G16Y 10/60; G16Y 20/40

(57) ABSTRACT

An internet of things (IoT) based emergency medical event management system for identifying emergency medical events and providing rescue medication during the emergency medical events of individuals, is provided. The IoT-based emergency medical event management system includes a non-invasive device configured to collect, using sensors, at least one of: biological samples and biomarkers, from the individuals. The non-invasive device is further configured to process the data associated with at least one of: the biological samples and the biomarkers, to detect severity of the emergency medical events. The IoT-based emergency medical event management system further includes a communication device configured to analyze information associated with the severity of the emergency medical events. The IoT-based emergency medical event management system further includes an IoT-enabled drug delivery device configured to provide a corresponding rescue medication based on the severity of the emergency medical events using drug delivery mechanisms.

### EARLIEST PRIORITY DATE

This application claims priority from a Provisional patent application filed in the United States of America having Patent Application No. 63/590,791, on Oct. 17, 2023, and titled “DEVICE FOR NON-INVASIVE EARLY IDENTIFICATION OF MEDICAL CONDITIONS ANDINTERNET OF THINGS ENABLED DRUG DELIVERY”.

### TECHNICAL FIELD

Embodiments of the present disclosure relate to internet of things (IoT) based medical devices, and more particularly relate to an IoT-based emergency medical event management system for identifying one or more emergency medical events and providing rescue medication responding to the one or more emergency medical events of one or more individuals.

### BACKGROUND

In recent years, advancements in medical device technology and Internet of Things (IoT) integration have led to significant improvements in the field of healthcare. One notable trend is the development of non-invasive diagnostic devices and IoT-enabled drug delivery systems.

Non-invasive diagnostic devices have gained prominence due to their ability to provide valuable health information without the need for invasive procedures. These non-invasive diagnostic devices employ a range of technologies, including sensors, imaging, and biomarker analysis, to detect and monitor medical conditions. They have been instrumental in early disease detection, enabling more timely interventions and improved outcomes for individuals. For example, non-invasive diagnostic tools have been developed for conditions like diabetes, where continuous glucose monitoring systems allow individuals to track their blood sugar levels without requiring frequent fingerstick tests. In the field of cardiology, wearable devices equipped with ECG sensors may monitor heart rhythms and detect arrhythmias, offering early warning signs of potential cardiac issues.

Integrating IoT technology into healthcare devices has transformed how individuals and healthcare professionals manage and monitor medical conditions. IoT-enabled devices collect real-time data, transmit the collected data securely to cloud-based platforms, and enable remote monitoring and management of the health of individuals. For instance, IoT-enabled insulin pumps for diabetes management adjust insulin delivery based on real-time glucose levels and even send alerts to caregivers or healthcare providers in case of critical events. These IoT-enabled devices improve individual compliance and enable personalized treatment plans.

Additionally, IoT platforms are used to create interconnected ecosystems of healthcare devices, allowing for seamless data sharing and coordination among various medical devices and systems. This integration enhances the overall quality of care and supports remote individual monitoring, especially valuable for individuals with chronic conditions.

While non-invasive diagnostics and IoT-enabled drug delivery systems have shown promise, there are still challenges to overcome. Ensuring the accuracy and reliability of diagnostic data is critical, as is addressing privacy and security concerns in IoT-connected healthcare devices. Regulatory compliance and standardization efforts continue to evolve to ensure safety and effectiveness of these technologies.

In existing technology, an electronic nose development and preliminary human breath testing for rapid, non-invasive COVID-19 detection are disclosed. The electronic nose (E-Nose) is designed for rapid on-site screening of COVID-19 infection by analyzing volatile organic compounds (VOCs) in exhaled human breath. The E-Nose consists of a hand-held E-Nose sensor system with 64 chemically sensitive nanomaterial sensing elements tailored to detect COVID-19 VOCs. The E-Nose includes data acquisition electronics, a tablet with control software, and a sampling fixture to capture breath samples. However, the E-Nose may lack specificity, as the E-Nose relies on VOC patterns that are influenced by a plurality of factors, potentially leading to false positives or negatives. The system associated with the E-Nose is relatively complex, requiring multiple components and Nano-sensors. This complexity limits the case of use and portability of the E-Nose compared to simpler non-invasive devices.

There are various technical problems with the non-invasive early identification of medical conditions and IoT-enabled drug delivery in the prior art. Many existing non-invasive diagnostic devices may lack the precision required for early identification of specific medical conditions. Variability in data accuracy and reliability may impact the effectiveness of diagnosis and treatment. Integration between different medical devices and platforms can be complex and problematic. Ensuring seamless data exchange and compatibility among various components in an IoT ecosystem is a technical hurdle. Detecting and treating serious or life-threatening events across a spectrum of medical conditions is a critical challenge in modern healthcare. Various conditions, including at least one of: seizures, myocardial infarction, diabetes-related hypoglycemia, sickle cell pain crises, cancer pain, opioid overdoses, benzodiazepine overdoses, and chemical exposures, require timely intervention to prevent adverse outcomes.

Therefore, there is a need for an improved IoT-based emergency medical event management system and method for identifying one or more emergency medical events and providing rescue medication responding to the one or more emergency medical events of one or more individuals, in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, an internet of things (IoT) based emergency medical event management system for identifying one or more emergency medical events and providing rescue medication during the one or more emergency medical events of one or more individuals, is provided. The IoT-based emergency medical event management system comprises one or more non-invasive devices comprise one or more sensors configured to collect at least one of: one or more biological samples and one or more biomarkers, from the one or more individuals. The one or more non-invasive devices further comprise one or more processors configured to: (a) obtain data associated with at least one of: the one or more biological samples and the one or more biomarkers, from the one or more sensors; and (b) process the data associated with at least one of: the one or more biological samples and the one or more biomarkers, to detect severity of the one or more emergency medical events.

The IoT-based emergency medical event management system further comprises one or more communication devices communicatively connected to the one or more non-invasive devices. The one or more communication devices are configured to: (a) obtain information associated with the severity of the one or more emergency medical events, from the one or more non-invasive devices; and (b) analyze the information associated with the severity of the one or more emergency medical events. The IoT-based emergency medical event management system further comprises one or more IoT-enabled drug delivery devices are communicatively connected to the one or more communication devices. The one or more IoT-enabled drug delivery devices are configured to: (a) obtain the analyzed information associated with the severity of the one or more emergency medical events, from the one or more communication devices; and (b) provide a corresponding rescue medication based on the severity of the one or more emergency medical events using one or more drug delivery mechanisms.

In an embodiment, in processing the data associated with at least one of: the one or more biological samples and the one or more biomarkers, to detect the severity of the one or more emergency medical events, the one or more processors of the one or more non-invasive devices are configured to: (a) obtain the data associated with at least one of: the one or more biological samples and the one or more biomarkers, from the one or more sensors of the one or more non-invasive devices, wherein the data associated with at least one of: the one or more biological samples and the one or more biomarkers, comprise concentration of biological compounds measured from the data associated with at least one of: the one or more biological samples and the one or more biomarkers, (b) compare the measured concentration of biological compounds, with one or more pre-determined threshold values, wherein the one or more pre-determined threshold values comprise one or more ranges indicating the severity of the one or more emergency medical events, and (c) detect the severity of the one or more emergency medical events based on determination of one or more ranges, upon comparison of the measured concentration of biological compounds, with the one or more pre-determined threshold values.

In another embodiment, the one or more sensors comprise at least one of: (a) a continuous glucose monitoring (CGM) sensor configured to detect glucose level in blood, (b) a breath sensor configured to detect at least one of: ethane, pentane, and relevant volatile organic compounds (VOC) corresponding to myocardial infarction, i (c) a wearable electroencephalogram (EEG) sensor configured to detect one or more electrical activities of a brain, (d) an oxygen saturation sensor configured to detect blood oxygen level, (c) an electrocardiogram sensor (ECG) configured to detect one or more electrical activities of a heart, and (f) one or more wearable glasses configured to monitor eye movements indicating at least one of: seizure and one or more neurological issues.

In yet another embodiment, the one or more processors of the one or more non-invasive devices are configured to: (a) generate one or more scores indicating a level of confidence for detecting the severity of the one or more emergency medical events, and (b) transmit information associated with the generated one or more scores indicating the level of confidence for detecting the severity of the one or more emergency medical events, to the one or more communication devices.

In yet another embodiment, the one or more communication devices are configured to: (a) obtain the information associated with the generated one or more scores indicating the level of confidence for detecting the severity of the one or more emergency medical events, from the one or more non-invasive devices, (b) analyze the severity of the one or more emergency medical events based on the generated one or more scores indicating the level of confidence, and (c) trigger one or more signals to activate the one or more IoT-enabled drug delivery devices for providing the corresponding rescue medication based on the severity of the one or more emergency medical events.

In yet another embodiment, the biological compounds measured from at least one of: the one or more biological samples and the one or more biomarkers, comprise one or more volatile organic compounds. The one or more volatile organic compounds are identified in at least one of: the one or more biological samples and the one or more biomarkers, using one or more gas sensors with chemical analysis techniques.

In one aspect, an internet of things (IoT) based emergency medical event management method for identifying one or more emergency medical events and providing rescue medication during the one or more emergency medical events of one or more individuals, is disclosed. The IoT-based emergency medical event management method comprises collecting, by one or more sensors of one or more non-invasive devices, at least one of: one or more biological samples and one or more biomarkers, from the one or more individuals.

The IoT-based emergency medical event management method further comprises obtaining, by one or more processors of the one or more non-invasive devices, data associated with at least one of: the one or more biological samples and the one or more biomarkers, from the one or more sensors. The IoT-based emergency medical event management method further comprises processing, by the one or more processors of the one or more non-invasive devices, the data associated with at least one of: the one or more biological samples and the one or more biomarkers, to detect severity of the one or more emergency medical events.

The IoT-based emergency medical event management method further comprises obtaining, by one or more communication devices, information associated with the severity of the one or more emergency medical events, from the one or more non-invasive devices. The IoT-based emergency medical event management method further comprises analyzing, by the one or more communication devices, the information associated with the severity of the one or more emergency medical events. The IoT-based emergency medical event management method further comprises obtaining, by one or more IoT-enabled drug delivery devices, the analyzed information associated with the severity of the one or more emergency medical events, from the one or more communication devices. The IoT-based emergency medical event management method further comprises providing, by the one or more IoT-enabled drug delivery devices, a corresponding rescue medication based on the severity of the one or more emergency medical events using one or more drug delivery mechanisms.

In another aspect, a non-transitory computer-readable storage medium having instructions stored therein that, when executed by a hardware processor, causes the processor to perform method steps as described above.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a block diagram illustrating a computing environment with an Internet of Things (IoT) based emergency medical event management system for identifying one or more emergency medical events and providing rescue medication responding to the one or more emergency medical events of one or more individuals, in accordance with an embodiment of the present disclosure;

FIG. 2 is a detailed view of one or more non-invasive devices for early identification of the one or more emergency medical events, in accordance with an embodiment of the present disclosure;

FIG. 3 illustrates an exemplary view of the one or more non-invasive devices for early identification of the one or more emergency medical events, in accordance with an embodiment of the present disclosure; and

FIG. 4 illustrates a flow chart illustrating an IoT-based emergency medical event management method for identifying the one or more emergency medical events and providing the rescue medication responding to the one or more emergency medical events of the one or more individuals, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

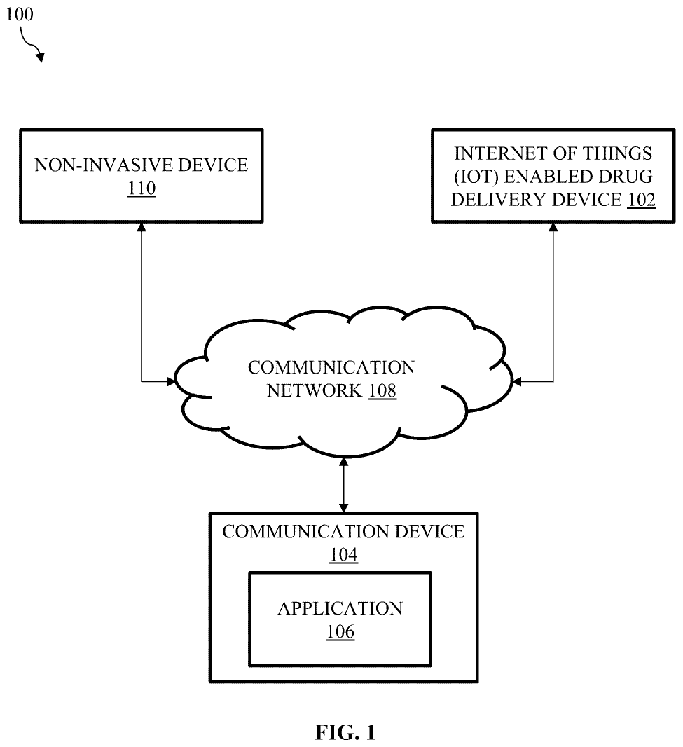

FIG. 1 is a block diagram illustrating a computing environment with an internet of things (IoT) based emergency medical event management system 100 for identifying one or more emergency medical events and providing rescue medication responding to the one or more emergency medical events of one or more individuals, in accordance with an embodiment of the present disclosure.

As shown in FIG. 1, the IoT-based emergency medical event management system 100 includes one or more non-invasive devices 110 works in conjunction with one or more communication devices 104 and one or more IoT-enabled drug delivery devices 102 through a communication network 108. In an embodiment, the terms “one or more emergency medical events” and “one or more critical medical events” are used interchangeably. The one or more non-invasive devices 110 are configured to collect at least one of: one or more biological samples and one or more biomarkers, from the one or more individuals, using one or more sensors. The one or more non-invasive devices 110 are configured to obtain data associated with at least one of: the one or more biological samples and the one or more biomarkers, from the one or more sensors, using one or more processors.

The one or more non-invasive devices 110 are further configured to process the data associated with at least one of: the one or more biological samples and the one or more biomarkers, to detect severity of the one or more emergency medical events, using the one or more processors.

The one or more communication devices 104 are communicatively connected to the one or more non-invasive devices 110. The one or more communication devices 104 are configured to obtain information associated with the severity of the one or more emergency medical events, from the one or more non-invasive devices 110. The one or more communication devices 104 are further configured to analyze the information associated with the severity of the one or more emergency medical events.

The one or more communication devices 104 may be digital devices, computing devices and/or networks. The one or more communication devices 104 may include, but is not limited to, a mobile device, a smartphone, a personal digital assistant (PDA), a tablet computer, a phablet computer, a wearable computing device, a laptop, a desktop, a server, and the like. The communication network 108 may be a wired communication network and/or a wireless communication network, a local area network (LAN), a wide area network (WAN), a metropolitan area network (MAN), a telephone network, such as the public switched telephone network (PSTN) or a cellular network, an intranet, an internet, a fiber optic network, a satellite network, a cloud computing network, or a combination of networks.

The one or more IoT-enabled drug delivery devices 102 are communicatively connected to the one or more communication devices 104 through the communication network 108. The one or more IoT-enabled drug delivery devices 102 are configured to obtain the analyzed information associated with the severity of the one or more emergency medical events, from the one or more communication devices 104. The one or more IoT-enabled drug delivery devices 102 are configured to provide a corresponding rescue medication based on the severity of the one or more emergency medical events using one or more drug delivery mechanisms.

In an embodiment, the one or more emergency medical events may include but not limited to seizures, myocardial infarction, diabetes-related hypoglycemia, sickle cell pain crises, cancer pain, opioid overdoses, benzodiazepine overdoses, chemical exposures, and the like. In an exemplary embodiment, an application 106 (e.g., a mobile application) is configured to be accessible to one or more users/individuals on the one or more communication devices 104. Integration of the application 106 with the one or more IoT-enabled drug delivery devices 102 or the one or more non-invasive devices 110 enhances the overall user experience, making the system easier for the one or more users/individuals to manage their health, and receive timely assistance during the one or more emergency medical events. The application 106 is adapted to facilitate data visualization and display information collected by the one or more communication devices 104. The information includes the severity of the one or more emergency medical events obtained from the one or more non-invasive devices 110, on the one or more communication devices 104.

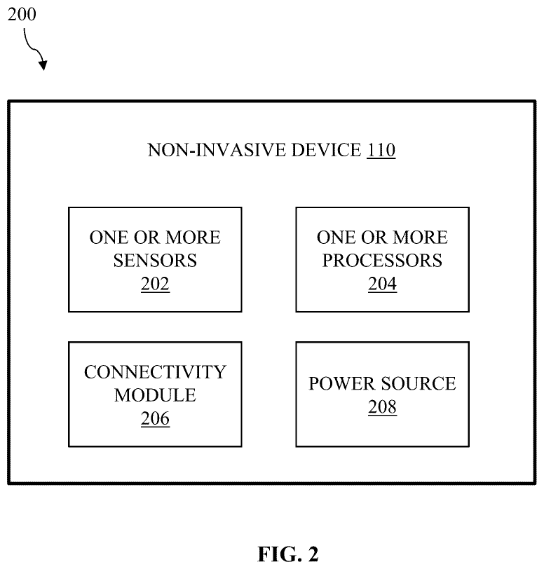

FIG. 2 is a detailed view 200 of one or more non-invasive devices 110 for early identification of the one or more emergency medical events, in accordance with an embodiment of the present disclosure.

The one or more non-invasive devices 110 include the one or more sensors 202, the one or more processors 204 (i.e., one or more hardware processors 204), a connectivity module 206, and a power source 208. The one or more sensors 202 are configured to collect at least one of: the one or more biological samples and the one or more biomarkers, from the one or more individuals. In an embodiment, the one or more biological samples may include one or more volatile organic compounds (VOCs) (e.g., ethane and pentane) and gases (i.e., gases associated with metabolic or respiratory conditions). In an embodiment, the one or more sensors 202 are configured with zinc oxide nanoparticles to measure relevant breath compounds accurately.

The one or more sensors 202 may include at least one of: a continuous glucose monitoring sensor, a breath sensor, a wearable electroencephalogram (EEG) sensor, an oxygen saturation sensor, an electrocardiogram sensor, and the like. The continuous glucose monitoring sensor is configured to detect glucose level in blood. The continuous glucose monitoring sensor may continuously monitor glucose levels and may apply a customizable threshold for intervention, such as administering glucagon at 70 mg/dL to prevent hypoglycaemic symptoms from occurring at 60 mg/dL.

The breath sensor is configured to detect at least one of: ethane, pentane, and relevant volatile organic compounds (VOC) corresponding to myocardial infarction. The wearable electroencephalogram (EEG) sensor configured to detect one or more electrical activities of a brain. The oxygen saturation sensor is configured to detect blood oxygen level. The electrocardiogram sensor (ECG) is configured to detect one or more electrical activities of a heart. Additionally, one or more wearable glasses tracking eye movements may also play a role in detecting conditions like seizures or neurological issues. In an embodiment, each of the one or more sensors/devices 202 may use its own detection algorithms (e.g., AI, threshold comparisons) to continuously the data including at least one of: glucose levels, brainwave patterns, VOCs, heart rhythms, and the like, based on at least one of: the one or more biological samples and the one or more biomarkers, collected from the one or more individuals.

The one or more processors 204 are configured to obtain the data associated with at least one of: the one or more biological samples and the one or more biomarkers, from the one or more sensors 202. The one or more processors 204 are further configured to process the data associated with at least one of: the one or more biological samples and the one or more biomarkers, to detect severity of the one or more emergency medical events.

For processing the data associated with at least one of: the one or more biological samples and the one or more biomarkers, to detect the severity of the one or more emergency medical events, the one or more processors 204 of the one or more non-invasive devices 110 are initially configured to obtain the data associated with at least one of: the one or more biological samples and the one or more biomarkers, from the one or more sensors 202 of the one or more non-invasive devices 110. In an embodiment, the data associated with at least one of: the one or more biological samples and the one or more biomarkers, may include concentration of biological compounds measured from the data associated with at least one of: the one or more biological samples and the one or more biomarkers.

The one or more processors 204 of the one or more non-invasive devices 110 are further configured to compare the measured concentration of biological compounds, with one or more pre-determined threshold values. The one or more pre-determined threshold values may include one or more ranges indicating the severity of the one or more emergency medical events. For example, the one or more processors 204 are configured to detect the MI by identifying the presence of biological compounds including at least one of: ethane and pentane in breath samples, based on one or more algorithms.

In an embodiment, the biological compounds measured from at least one of: the one or more biological samples and the one or more biomarkers, may include one or more volatile organic compounds. In an embodiment, the one or more volatile organic compounds are identified in at least one of: the one or more biological samples and the one or more biomarkers, using one or more gas sensors with chemical analysis techniques.

The one or more processors 204 of the one or more non-invasive devices 110 are further configured to detect the severity of the one or more emergency medical events based on determination of the one or more ranges, upon comparison of the measured concentration of biological compounds, with the one or more pre-determined threshold values. For example, if the one or more ranges may be determined as one or more first ranges, then the one or more processors 204 are configured to detect the severity of the one or more emergency medical events, as healthy. In another example, if the one or more ranges may be determined as one or more second ranges, then the one or more processors 204 are configured to detect the severity of the one or more emergency medical events, as coronary artery diseases. In another example, if the one or more ranges may be determined as one or more second ranges, then the one or more processors 204 are configured to detect the severity of the one or more emergency medical events, as myocardial infarction (MI).

In an embodiment, the one or more processors 204 of the one or more non-invasive devices 110 are configured to generate one or more scores indicating a level of confidence for detecting the severity of the one or more emergency medical events. The one or more processors 204 of the one or more non-invasive devices 110 are further configured to transmit information associated with the generated one or more scores indicating the level of confidence for detecting the severity of the one or more emergency medical events, to the one or more communication devices 104. In other words, the one or more processors 204 of the one or more non-invasive devices 110 are configured to provide a binary diagnosis (positive/negative) or a probability score indicating the level of confidence in the diagnosis in the one or more communication devices 104. In an embodiment, the one or more non-invasive devices 110 may provide user feedback through the application 106 associated with the one or more communication devices 104.

The one or more communication devices 104 are configured to obtain the information associated with the generated one or more scores indicating the level of confidence for detecting the severity of the one or more emergency medical events, from the one or more non-invasive devices 110. The one or more communication devices 104 are further configured to analyze the severity of the one or more emergency medical events based on the generated one or more scores indicating the level of confidence. The one or more communication devices 104 are further configured to trigger one or more signals to activate the one or more IoT-enabled drug delivery devices 102 for providing the corresponding rescue medication based on the severity of the one or more emergency medical events using the one or more drug delivery mechanisms in the one or more IoT-enabled drug delivery devices 102.

In an embodiment, the one or more drug delivery mechanisms may be dual drug delivery mechanism (e.g., automatic mode and manual mode) in the one or more IoT-enabled drug delivery devices 102, based on the preference of the one or more individual and the severity of the one or more emergency medical events. In an embodiment, the one or more drug delivery mechanisms are configured to automatically inject the corresponding rescue medication subcutaneously in response to the detection of the one or more emergency medical events occurred to the one or more individuals.

In another embodiment, the one or more IoT-enabled drug delivery devices 102 are configured to inject the corresponding rescue medication subcutaneously by pressing one or more control buttons (in the manual mode) by at least one of: the one or more individuals and one or more users during the one or more emergency medical events, to operate the one or more drug delivery mechanisms. In such a case, the one or more control buttons are adapted to be pressed by the one or more users/individuals during the emergency medical event, thereby administering the rescue medication to the individual subcutaneously.

The connectivity module 206 is configured to transmit data associated with the one or more emergency medical events, to the one or more communication devices 104 through the communication network 108. The connectivity module 206 is configured to provide real-time data transmission. The power source 208 in the one or more non-invasive devices 110 or in the one or more IoT-enabled drug delivery devices 102, may be a rechargeable battery or a replaceable one, depending on the intended usage duration of the one or more non-invasive devices 110 or the one or more IoT-enabled drug delivery devices 102. Energy-efficient power management is crucial, especially for the one or more non-invasive devices 110 or the one or more IoT-enabled drug delivery devices 102.

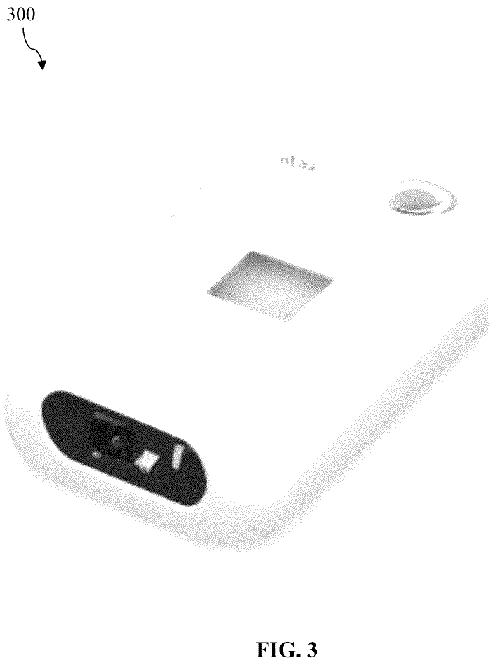

FIG. 3 illustrates an exemplary view 300 of the one or more non-invasive devices 110 for early identification of the one or more emergency medical events, in accordance with an embodiment of the present disclosure. FIG. 3 depicts arrangements of one or more parts of the one or more non-invasive devices 110, including at least one of: the one or more sensors 202, the one or more processors 204, the connectivity module 206, and the power source 208. The detailed explanation for the above said parts of the one or more non-invasive devices 110 have been described in FIG. 2.

In an exemplary embodiment, the one or more non-invasive devices 110 are configured for non-invasive diagnosis, specifically focusing on myocardial infarction (MI) but with broader applications. The one or more non-invasive devices 110 are configured to allow the one or more individuals to diagnose the MI and potentially other conditions by simply breathing into the one or more non-invasive devices 110. The one or more non-invasive devices 110 are configured with the one or more sensors 202 with the zinc oxide nanoparticles to measure relevant breath compounds accurately. The one or more sensors 202 are capable of distinguishing breath signatures between MI individuals, coronary artery disease individuals, and healthy individuals with high accuracy, based on at least one of: the one or more biological samples and the one or more biomarkers, collected from the one or more individuals. The broader applications of the one or more non-invasive devices 110 include IoT integration, where the one or more non-invasive devices 110 are operatively connected to the one or more communication devices 104 and the one or more IoT-enabled drug delivery devices 102 for immediate and precise treatment post-diagnosis.

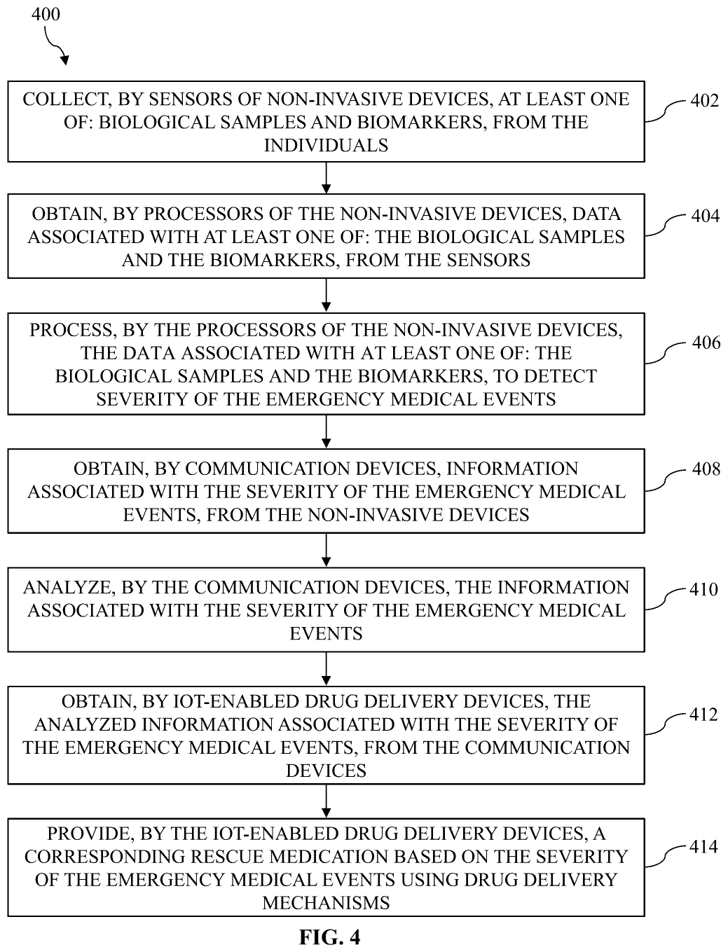

FIG. 4 illustrates a flow chart illustrating an IoT-based emergency medical event management method 400 for identifying the one or more emergency medical events and providing the rescue medication responding to the one or more emergency medical events of the one or more individuals, in accordance with an embodiment of the present disclosure.

At step 402, at least one of: the one or more biological samples and the one or more biomarkers, are collected, using the one or more sensors 202, from the one or more individuals.

At step 404, the data associated with at least one of: the one or more biological samples and the one or more biomarkers, are obtained by the one or more processors 204 from the one or more sensors 202.

At step 406, the data associated with at least one of: the one or more biological samples and the one or more biomarkers, are processed by the one or more processors 204 to detect the severity of the one or more emergency medical events.

At step 408, the information associated with the severity of the one or more emergency medical events, are obtained by the one or more communication devices 104 from the one or more non-invasive devices 110.

At step 410, the information associated with the severity of the one or more emergency medical events, are analyzed by the one or more communication devices 104.

At step 412, the analyzed information associated with the severity of the one or more emergency medical events, are obtained by the one or more IoT-enabled drug delivery devices 102 from the one or more communication devices 104.

At step 414, the corresponding rescue medication is provided by the one or more IoT-enabled drug delivery devices 102, based on the severity of the one or more emergency medical events using one or more drug delivery mechanisms.

Numerous advantages of the present disclosure may be apparent from the discussion above. In accordance with the present disclosure, the IoT-based emergency medical event management system 100 for early identification of the one or more emergency medical conditions and provision of the rescue medication responding to the one or more emergency medical events of one or more individuals, are disclosed. By utilizing non-invasive diagnostic techniques, the IoT-based emergency medical event management system 100 eliminates the need for invasive and uncomfortable procedures, enhancing individual comfort and compliance. The IoT-based emergency medical event management system 100 with the one or more non-invasive devices 110 and the one or more IoT-enabled drug delivery devices 102, enables the early detection of the one or more critical medical conditions, allowing for timely intervention and treatment initiation.

The one or more IoT-enabled drug delivery devices 102 facilitate personalized treatment plans by tailoring drug delivery to the individual's specific condition, ensuring the right medications are administered at the right time. Integration with IoT technology enables real-time data transmission, remote monitoring, and communication with healthcare providers, enhancing the quality of care and patient outcomes. The one or more non-invasive devices 110 and the one or more IoT-enabled drug delivery devices 102, facilitate an intuitive and user-friendly interface, making it accessible to the one or more individuals without extensive medical training. By swiftly identifying the one or more critical medical events and initiating prompt treatment, the one or more non-invasive devices 110 and the one or more IoT-enabled drug delivery devices 102 have the potential to save lives, reduce healthcare costs, and improve overall health outcomes.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer-readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the IoT-based emergency medical event management system 100 either directly or through intervening I/O controllers. Network adapters may also be coupled to the IoT-based emergency medical event management system 100 to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/IoT-based emergency medical event management system 100 in accordance with the embodiments herein. The IoT-based emergency medical event management system 100 herein comprises at least one processor or central processing unit (CPU). The CPUs are interconnected via a system bus to various devices including at least one of: a random-access memory (RAM), read-only memory (ROM), and an input/output (I/O) adapter. The I/O adapter can connect to peripheral devices, including at least one of: disk units and tape drives, or other program storage devices that are readable by the IoT-based emergency medical event management system 100. The IoT-based emergency medical event management system 100 can read the inventive instructions on the program storage devices and follow these instructions to execute the methodology of the embodiments herein.

The IoT-based emergency medical event management system 100 further includes a user interface adapter that connects a keyboard, mouse, speaker, microphone, and/or other user interface device including a touch screen device (not shown) to the bus to gather user input. Additionally, a communication adapter connects the bus to a data processing network, and a display adapter connects the bus to a display device which may be embodied as an output device including at least one of: a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that are issued on an application based here on. Accordingly, the embodiments of the present invention are intended to be illustrative, but not limiting, of the scope of the invention, which is set forth in the following claims.

What is claimed is:

1. An Internet of Things (IoT) based emergency medical event management system for identifying one or more emergency medical events and providing rescue medication during the one or more emergency medical events of one or more individuals, the non-invasive based emergency medical event management system comprising:
one or more non-invasive devices, wherein the one or more non-invasive devices comprise:
one or more sensors configured to collect at least one of: one or more biological samples and one or more biomarkers, from the one or more individuals;
one or more processors configured to:
obtain data associated with at least one of: the one or more biological samples and the one or more biomarkers, from the one or more sensors; and
process the data associated with at least one of: the one or more biological samples and the one or more biomarkers, to detect severity of the one or more emergency medical events;
one or more communication devices communicatively connected to the one or more non-invasive devices, wherein the one or more communication devices are configured to:
obtain information associated with the severity of the one or more emergency medical events, from the one or more non-invasive devices; and
analyze the information associated with the severity of the one or more emergency medical events; and
one or more IoT-enabled drug delivery devices are communicatively connected to the one or more communication devices, wherein the one or more IoT-enabled drug delivery devices are configured to:
obtain the analyzed information associated with the severity of the one or more emergency medical events, from the one or more communication devices; and
provide a corresponding rescue medication based on the severity of the one or more emergency medical events using one or more drug delivery mechanisms.

2. The IoT-based emergency medical event management system of claim 1, wherein in processing the data associated with at least one of: the one or more biological samples and the one or more biomarkers, to detect the severity of the one or more emergency medical events, the one or more processors of the one or more non-invasive devices are configured to:
obtain the data associated with at least one of: the one or more biological samples and the one or more biomarkers, from the one or more sensors of the one or more non-invasive devices, wherein the data associated with at least one of: the one or more biological samples and the one or more biomarkers, comprise concentration of biological compounds measured from the data associated with at least one of: the one or more biological samples and the one or more biomarkers;
compare the measured concentration of biological compounds, with one or more pre-determined threshold values, wherein the one or more pre-determined threshold values comprise one or more ranges indicating the severity of the one or more emergency medical events; and
detect the severity of the one or more emergency medical events based on determination of one or more ranges, upon comparison of the measured concentration of biological compounds, with the one or more pre-determined threshold values.

3. The IoT-based emergency medical event management system of claim 1, wherein the one or more sensors comprise at least one of:
a continuous glucose monitoring (CGM) sensor configured to detect glucose level in blood;
a breath sensor configured to detect at least one of: ethane, pentane, and relevant volatile organic compounds (VOC) corresponding to myocardial infarction;
a wearable electroencephalogram (EEG) sensor configured to detect one or more electrical activities of a brain;
an oxygen saturation sensor configured to detect blood oxygen level;
an electrocardiogram sensor (ECG) configured to detect one or more electrical activities of a heart; and
one or more wearable glasses configured to monitor eye movements indicating at least one of: seizure and one or more neurological issues.

4. The IoT-based emergency medical event management system of claim 1, wherein the one or more processors of the one or more non-invasive devices are configured to:
generate one or more scores indicating a level of confidence for detecting the severity of the one or more emergency medical events; and
transmit information associated with the generated one or more scores indicating the level of confidence for detecting the severity of the one or more emergency medical events, to the one or more communication devices.

5. The IoT-based emergency medical event management system of claim 4, wherein the one or more communication devices are configured to:
obtain the information associated with the generated one or more scores indicating the level of confidence for detecting the severity of the one or more emergency medical events, from the one or more non-invasive devices;
analyze the severity of the one or more emergency medical events based on the generated one or more scores indicating the level of confidence; and
trigger one or more signals to activate the one or more IoT-enabled drug delivery devices for providing the corresponding rescue medication based on the severity of the one or more emergency medical events.

6. The IoT-based emergency medical event management system of claim 2, wherein the biological compounds measured from at least one of: the one or more biological samples and the one or more biomarkers, comprise one or more volatile organic compounds, and
wherein the one or more volatile organic compounds are identified in at least one of: the one or more biological samples and the one or more biomarkers, using one or more gas sensors with chemical analysis techniques.

7. An internet of things (IoT) based emergency medical event management method for identifying one or more emergency medical events and providing rescue medication during the one or more emergency medical events of one or more individuals, the IoT-based emergency medical event management method comprising:
collecting, by one or more sensors of one or more non-invasive devices, at least one of: one or more biological samples and one or more biomarkers, from the one or more individuals;
obtaining, by one or more processors of the one or more non-invasive devices, data associated with at least one of: the one or more biological samples and the one or more biomarkers, from the one or more sensors;
processing, by the one or more processors of the one or more non-invasive devices, the data associated with at least one of: the one or more biological samples and the one or more biomarkers, to detect severity of the one or more emergency medical events;
obtaining, by one or more communication devices, information associated with the severity of the one or more emergency medical events, from the one or more non-invasive devices;
analyzing, by the one or more communication devices, the information associated with the severity of the one or more emergency medical events;
obtaining, by one or more IoT-enabled drug delivery devices, the analyzed information associated with the severity of the one or more emergency medical events, from the one or more communication devices; and
providing, by the one or more IoT-enabled drug delivery devices, a corresponding rescue medication based on the severity of the one or more emergency medical events using one or more drug delivery mechanisms.

8. The IoT-based emergency medical event management method of claim 7, wherein processing the data associated with at least one of: the one or more biological samples and the one or more biomarkers, to detect the severity of the one or more emergency medical events, comprising:
obtaining, by the one or more processors of the one or more non-invasive devices, the data associated with at least one of: the one or more biological samples and the one or more biomarkers, from the one or more sensors of the one or more non-invasive devices, wherein the data associated with at least one of: the one or more biological samples and the one or more biomarkers, comprise concentration of biological compounds measured from the data associated with at least one of: the one or more biological samples and the one or more biomarkers;
comparing, by the one or more processors of the one or more non-invasive devices, the measured concentration of biological compounds, with one or more pre-determined threshold values, wherein the one or more pre-determined threshold values comprise one or more ranges indicating the severity of the one or more emergency medical events; and
detecting, by the one or more processors of the one or more non-invasive devices, the severity of the one or more emergency medical events based on determination of one or more ranges, upon comparison of the measured concentration of biological compounds, with the one or more pre-determined threshold values.

9. The IoT-based emergency medical event management method of claim 7, wherein collecting at least one of: the one or more biological samples and the one or more biomarkers, from the one or more individuals, comprises:
collecting, by a continuous glucose monitoring sensor, at least one of: the one or more biological samples and the one or more biomarkers, to detect glucose level in a blood;
collecting, by a breath sensor, at least one of: the one or more biological samples and the one or more biomarkers, to detect at least one of: ethane, pentane, and relevant volatile organic compounds (VOC) corresponding to myocardial infarction;
collecting, by a wearable electroencephalogram (EEG) sensor, at least one of: the one or more biological samples and the one or more biomarkers, to detect one or more electrical activities of a brain;
collecting, by an oxygen saturation sensor, at least one of: the one or more biological samples and the one or more biomarkers, to detect blood oxygen level;
collecting, by an electrocardiogram (ECG) sensor, at least one of: the one or more biological samples and the one or more biomarkers, to detect one or more electrical activities of a heart; and
collecting, by one or more wearable glasses, information associated with eye movements to detect at least one of: seizure and one or more neurological issues.

10. The IoT-based emergency medical event management method of claim 7, further comprising:
generating, by the one or more processors of the one or more non-invasive devices, one or more scores indicating a level of confidence for detecting the severity of the one or more emergency medical events; and
transmitting, by the one or more processors of the one or more non-invasive devices, information associated with the generated one or more scores indicating the level of confidence for detecting the severity of the one or more emergency medical events, to the one or more communication devices.

11. The IoT-based emergency medical event management method of claim 10, further comprising:
obtaining, by the one or more communication devices, the information associated with the generated one or more scores indicating the level of confidence for detecting the severity of the one or more emergency medical events, from the one or more non-invasive devices;
analyzing, by the one or more communication devices, the severity of the one or more emergency medical events based on the generated one or more scores indicating the level of confidence; and
triggering, by the one or more communication devices, one or more signals to activate the one or more IoT-enabled drug delivery devices for providing the corresponding rescue medication based on the severity of the one or more emergency medical events.

12. The IoT-based emergency medical event management method of claim 8, wherein the biological compounds measured from at least one of: the one or more biological samples and the one or more biomarkers, comprise one or more volatile organic compounds, and
wherein the one or more volatile organic compounds are identified in at least one of: the one or more biological samples and the one or more biomarkers, using one or more gas sensors with chemical analysis techniques.

13. A non-transitory computer-readable storage medium having instructions stored therein that when executed by one or more hardware processors, cause the one or more hardware processors to execute operations of:
collecting at least one of: one or more biological samples and one or more biomarkers, from the one or more individuals;
obtaining data associated with at least one of: the one or more biological samples and the one or more biomarkers, at one or more non-invasive devices, from the one or more sensors;
processing the data associated with at least one of: the one or more biological samples and the one or more biomarkers, to detect severity of the one or more emergency medical events, using one or more processors of the one or more non-invasive devices;
obtaining information associated with the severity of the one or more emergency medical events, at one or more communication devices, from the one or more non-invasive devices;
analyzing the information associated with the severity of the one or more emergency medical events, using the one or more communication devices;
obtaining the analyzed information associated with the severity of the one or more emergency medical events, at one or more IoT-enabled drug delivery devices, from the one or more communication devices; and
providing a corresponding rescue medication based on the severity of the one or more emergency medical events using one or more drug delivery mechanisms of the one or more IoT-enabled drug delivery devices.

14. The non-transitory computer-readable storage medium of claim 13, wherein processing the data associated with at least one of: the one or more biological samples and the one or more biomarkers, to detect the severity of the one or more emergency medical events, comprising:
obtaining the data associated with at least one of: the one or more biological samples and the one or more biomarkers, from the one or more sensors of the one or more non-invasive devices, wherein the data associated with at least one of: the one or more biological samples and the one or more biomarkers, comprise concentration of biological compounds measured from the data associated with at least one of: the one or more biological samples and the one or more biomarkers;
comparing the measured concentration of biological compounds, with one or more pre-determined threshold values, wherein the one or more pre-determined threshold values comprise one or more ranges indicating the severity of the one or more emergency medical events; and
detecting the severity of the one or more emergency medical events based on determination of one or more ranges, upon comparison of the measured concentration of biological compounds, with the one or more pre-determined threshold values.

15. The non-transitory computer-readable storage medium of claim 13, wherein collecting at least one of: the one or more biological samples and the one or more biomarkers, from the one or more individuals, comprises:
collecting at least one of: the one or more biological samples and the one or more biomarkers, to detect glucose level in a blood using a continuous glucose monitoring sensor;
collecting at least one of: the one or more biological samples and the one or more biomarkers, to detect at least one of: ethane, pentane, and relevant volatile organic compounds (VOC) corresponding to myocardial infarction, using a breath sensor;
collecting at least one of: the one or more biological samples and the one or more biomarkers, to detect one or more electrical activities of a brain, using a wearable electroencephalogram (EEG) sensor;
collecting at least one of: the one or more biological samples and the one or more biomarkers, to detect blood oxygen level, using an oxygen saturation sensor;
collecting at least one of: the one or more biological samples and the one or more biomarkers, to detect one or more electrical activities of a heart, using an electrocardiogram (ECG) sensor; and
collecting information associated with eye movements to detect at least one of: seizure and one or more neurological issues, using one or more wearable glasses.

16. The non-transitory computer-readable storage medium of claim 13, further comprising:
generating one or more scores indicating a level of confidence for detecting the severity of the one or more emergency medical events, using the one or more processors of the one or more non-invasive devices; and
transmitting information associated with the generated one or more scores indicating the level of confidence for detecting the severity of the one or more emergency medical events, to the one or more communication devices.

17. The non-transitory computer-readable storage medium of claim 16, further comprising:
obtaining the information associated with the generated one or more scores indicating the level of confidence for detecting the severity of the one or more emergency medical events, at the one or more communication devices, from the one or more non-invasive devices;
analyzing the severity of the one or more emergency medical events based on the generated one or more scores indicating the level of confidence, using the one or more communication devices; and
triggering one or more signals, using the one or more communication devices, to activate the one or more IoT-enabled drug delivery devices for providing the corresponding rescue medication based on the severity of the one or more emergency medical events.

18. The non-transitory computer-readable storage medium of claim 14, wherein the biological compounds measured from at least one of: the one or more biological samples and the one or more biomarkers, comprise one or more volatile organic compounds, and
wherein the one or more volatile organic compounds are identified in at least one of: the one or more biological samples and the one or more biomarkers, using one or more gas sensors with chemical analysis techniques.
